(19) United States
(12) Patent Application Publication
(10) Pub. No.: US 2015/0231295 A1
(43) Pub. Date: Aug. 20, 2015

(54) AQUEOUS FRAGRANCE RELEASE GELS

(71) Applicant: DOW GLOBAL TECHNOLOGIES LLC, Midland, MI (US)

(72) Inventors: Yonnie D. Yun, Shanghai (CN); Xin P. Jiang, Shanghai (CN)

(73) Assignee: Dow Global Technologies LLC, Midland, MI (US)

(21) Appl. No.: 14/434,804

(22) PCT Filed: Oct. 26, 2012
(86) PCT No.: PCT/CN2012/083598

§ 371 Date: Apr. 10, 2015

(51) Int. Cl. A61L 9/012; C11B 9/00

(52) U.S. Cl. CPC A61L 9/012; C11B 9/00

(57) ABSTRACT

Described are aqueous gel compositions for forming a fragrance-containing gels, comprising a gel network blend, comprising a) methylcellulose, hydroxypropyl methylcellulose, carboxymethylcellulose, or mixtures thereof, and b) alginic acid, alginates, or mixtures thereof, in a weight ratio ratio of from 1:8 to 8:1; from 1 to 40 wt. % of a fragrance oil; and from 0.01 to 10 wt. % of a salt, thereby cross-linking the alginic acid, alginates, or mixtures thereof; provided that fragrance-containing gel is heat resistant up to 50° C.

### FIELD

The present invention relates to a home care compositions, and methods for controlled fragrance release.

### BACKGROUND

In the home care field, there is a need for materials which encapsulate or entrain fragrance materials and then slowly release the fragrance to impart a pleasing scent. However, there are a number of considerations which demand attention from the industry, including a need for high active content, resistance to heat, appropriate hardness and elasticity, desirable water retention, and environmental compatibility.

Accordingly, what is needed are new fragrance release systems which address the above-described needs.

### DETAILED DESCRIPTION

In one embodiment, the present invention provides an aqueous gel composition for forming a fragrance-containing gel, comprising a gel network blend, comprising a) methylcellulose, hydroxypropyl methylcellulose, carboxymethylcellulose, or mixtures thereof, and b) alginic acid, alginates, or mixtures thereof, in a weight ratio ratio of from 1:8 to 8:1; from 1 to 40 wt. % of a fragrance oil; and from 0.01 to 10 wt. % of a salt, thereby cross-linking the alginic acid, alginates, or mixtures thereof; provided that fragrance-containing gel is heat resistant up to 50° C.

“Aqueous gel composition” refers to the fact that the major component is water. In one embodiment, at least 20% by weight of the aqueous gel composition (“wt. %”) is water, preferably at least 40 wt. %, more preferably at least 50 wt. %, more preferably at least 60 wt. %, more preferably at least 70 wt. %, more preferably at least 80 wt. %, up to 90 wt. %.

### Fragrance-Containing Gel

It is understood that the aqueous gel composition sets, cures, cross-links, or otherwise gels to form the fragrance-containing gel. Accordingly, the fragrance-containing gel may vary in hardness, but in any case, cannot be a liquid. In one embodiment, however, the fragrance-containing gel is finely divided and the resulting particles dispersed in any conventional home care formulation.

“Heat resistant” means that the gel experiences substantially no syneresis (contraction of a gel accompanied by loss of a liquid component of the gel). In one embodiment, the fragrance-containing gel is heat resistant up to at least 55° C., more preferably up to at least 60° C., more preferably up to at least 70° C., more preferably up to at least 80° C., and more preferably up to at least 90° C.

In some embodiments, the fragrance-containing gel is less than 20% dissolvable in water, preferably less than 10% dissolvable in water, preferably does not dissolve in water.

### Gel Network Blend

The gel network blend comprises a part a) comprising methylcellulose, hydroxypropyl methylcellulose, carboxymethylcellulose, or mixtures thereof.

In one embodiment, the methylcellulose has an average degree of substitution DS_{methyl} of from 1.2 to 2.0, more preferably from 1.5 to 1.9 and most preferably from 1.7 to 1.9. Typically, viscosities of 2% by weight aqueous methylcellulose solutions at 20° C., determined with a Brookfield viscometer, range from 40 to 80,000 mP·s, preferably from 1,000 to 78,000 mP·s, and more preferably from 15,000 to 75,000 mP·s. Examples of commercially available methylcelluloses that are useful in the present invention include METHOCEL™ A, SGA, E, K, and G series; especially preferred is METHOCEL™ A40M (DS_{methyl}=1.8, 2% by weight viscosity=40,000 mPa·s,) available from The Dow Chemical Company, Midland, U.S.A.

In one embodiment, the hydroxypropyl methylcellulose has an average degree of substitution DS_{methyl} of from 1.2 to 2.0, more preferably of from 1.3 to 1.8, and most preferably from 1.3 to 1.5 and a molar degree of substitution MS_{hydroxypropyl} of from 0.1 to 0.25, more preferably of from 0.15 to 0.25, and most preferably of from 0.20 to 0.23. Typically, viscosities of 2% by weight aqueous hydroxypropyl methylcellulose solutions at 20° C., determined with a Brookfield viscometer, range from 15 to 250,000 mPa·s, preferably from 450 to 200,000 mPa·s, and more preferably from 4,000 to 180,000 mPa·s. Examples of commercially available hydroxypropyl methylcelluloses that are useful in the present invention include METHOCEL™ K100M (DS_{methyl}=1.4, MS_{hydroxypropyl}=0.21, 2% by weight viscosity=100,000 mPa·s), and more preferably METHOCEL™ K15M (DS_{methyl}=1.4, MS_{hydroxypropyl}=0.21, 2% by weight viscosity=15,000 mP·s), available from The Dow Chemical Company, Midland, U.S.A.

In one embodiment, the carboxymethylcellulose has a molar degree of substitution MS_{carboxy} of from 0.5 to 1.2, more preferably of from 0.6 to 1.1, and most preferably of from 0.7 to 0.95. Typically, viscosities of 1% by weight aqueous carboxymethylcellulose solutions at 20° C., determined with a Brookfield viscometer, range from 20 to 50000 mPa·s, preferably from 500 to 2000 mPa·s, and more preferably from 2000 to 10000 mPa·s. Examples of commercially available carboxymethylcelluloses that are useful in the present invention include WALOCEL™ CRT 50000 PA (MS_{carboxy}=0.7, 1% by weight Brookfield viscosity=7000 mPa·s), and more preferably WALOCEL™ CRT 30000 (MS_{carboxy}=0.9, 1% by weight Brookfield viscosity=3500 mP·s), available from The Dow Chemical Company, Midland, U.S.A.

In some embodiments the composition is free of any cellulose derivative other than methylcellulose, hydroxypropyl methylcellulose, or carboxymethylcellulose.

Without intending to be bound by theory, it is believed that the gel network blend requires an acidic polysaccharide that can cross-link or precipitate with metal ions. Accordingly, in one embodiment, the gel network blend comprises a part b) comprising gellan, gelatin, pectin, carrageenan, alginic acid, alginates, or mixtures thereof. In one preferred embodiment, the gel network blend comprises a part b) comprising alginic acid, alginates, or mixtures thereof. Alginic acid is a linear copolymer of (1-4)-linked β-D-mannuronic acid (M-unit) and α-L-guluronic acid (G-unit) which units are linked together in different sequences or blocks. The monomers can appear in homopolymeric blocks of consecutive G-units (G-blocks), consecutive M-units (M-blocks), alternating M- and G-units (MG-blocks), or randomly organized blocks. Alginate is the salt of alginic acid, for example sodium and/or calcium alginate. Alginic acid/alginate are extracted from seaweeds, such as giant kelp (Macrocystis pyrifera).

In one embodiment, the gel network blend has a part a) to part b) weight ratio of from 1:8 to 8:1, preferably from 1:2 to 6:1, preferably 1:1 or alternatively, in some embodiments, 4:1.

In one embodiment, the gel network blend is preferably present in the from 0.5 to 5 wt. %, more preferably from 1 to 4 wt. %, and most preferably from 1.5 to 3 wt. %.

In one embodiment, the composition is substantially free of curdlan, guar gum, fenugreek gum, locust bean gum, konjac gum, agarose, or mixtures thereof. These are non-ionic and non-acidic polysaccharide hydrocolloids.

Alternatively, in a different embodiment, the composition may include non-ionic and non-acidic polysaccharide hydrocolloids, and contemplated are ratios of 1:9 to 10:1 alginate to non-ionic and non-acidic polysaccharide hydrocolloid, preferably 2:3 to 3:2.

### Salt

“Salt” refers to at least one inorganic cation. Preferably, the salt is a divalent cation such as for example Ca²⁺, Mg²⁺, and/or Zn²⁺ cations. Examples of suitable gel-promoting salts include calcium phosphate, calcium hydrogen phosphate, and mixtures thereof. If calcium phosphate, calcium hydrogen phosphate or another hydrogen phosphate or hydrogen phosphate of low solubility is used, glucono delta-lactone (GDL) may be added which gradually reacts with the (hydrogen) phosphate to release the cation. A preferred amount of GDL is within the range of from 0.01 to 2 wt. %. The salt is preferably from 0.01 to 5 wt. % of the aqueous gel composition, more preferably from 0.05 to 3 wt. %, more preferably from 0.1 to 2 wt. %, more preferably from 0.1 to 0.5 wt. %.

### Fragrance Oil

“Fragrance oil” include any hydrophobic component which provides a pleasant scent. Examples include scents that are floral, ambery, woody, leather, chypre, fougère, musk, vanilla, fruit, and/or citrus. Fragrance oils are obtained by extraction of natural substances or synthetically produced. Fragrances produced may be simple (one essence) or complex (a mélange of essences). Often, the fragrance oils are accompanied by auxiliary materials, such as fixatives, extenders, stabilizers and solvents. The fragrance oil is in an amount of from 1 to 60 wt. % of the aqueous gel composition, preferably from 10 to 40 wt. %, more preferably from 5 to 25 wt. %, and even more preferably from 9 to 22 wt. %.

Optional ingredients include those conventionally used in home care fragrance releasing compositions, often referred to as “air fresheners,” and such optional ingredients include waxes, antimicrobial agents, and dyes.

The composition according to the present invention may be prepared by several methods known in the art. One exemplary route is to first prepare separate colloidal solutions of each of the cellulose ether and the alginate and then combine those colloidal solutions and add further optional ingredients. Another exemplary route is to first dry mix the cellulose ether and the alginate and then prepare a colloidal solution of the mixture and add further optional ingredients. In either case, the resulting colloidal solution containing mixture typically gels within one to three hours.

In one embodiment, the composition is emulsified before gelling. Typically, the oil-containing compositions of the present invention form stable oil-in-water emulsions. In one embodiment, emulsifying salts may be added in addition to the gel-promoting salts described above. Examples of suitable emulsifying salts include trisodium phosphate, disodium hydrogen phosphate, sodium dihydrogen phosphate, sodium citrate, and alkaline sodium aluminum phosphate. Further exemplary ingredients that may be contained in the present composition include processing agents, such as emulsifiers different from emulsifying salts. In one embodiment, an emulsion is produced, and a portion of the emulsion dropped into a dipping solution (such as 5% CaCl₂). This produces hard capsules with entrained oil actives. In one embodiment, an emulsion is produced, and the emulsion is placed into a mold.

The compositions of the present invention have comparable physical properties to traditional gel products, including appearance, texture, rheology, and spreadability. They further exhibit outstanding thermal and refrigerating stability from −20 to 100° C., even with high moisture contents, as well as low syneresis. During their preparation no or only minimal undesired foaming is observed at high shear blending operations. Thus, the final product is largely bubble-free. In one embodiment, the high shear blending operations are carried out at room temperature.

The present composition can be manufactured with a wide range of texture rheology and elasticity. In some embodiments, compositions of the present invention form a stable gel from 5° C. to 95° C. In some embodiments, even at very high temperatures, the gel is still sliceable and moldable, which is very unique in hydrocolloids systems with such moisture content.

### EXAMPLES

### Example 1

Compositions of the present invention are described in Table 1, having the components listed in wt %.

TABLE 1

| | Batch | | | | | |
|---|---|---|---|---|---|---|
| | 1 | Batch 2 | Batch 3 | Batch 4 | Batch 5 | Batch 6 |
| METHOCEL | 1 | 1 | 1 | 1 | — | — |
| A4M | | | | | | |
| Methylcellulose | | | | | | |
| Carboxy | — | — | — | — | 1 | 1 |
| methylcellulose | | | | | | |
| Alginate | 1 | 1 | 1 | 1 | 1 | 1 |
| King Car | 20 | — | — | — | — | — |
| Fragrance | | | | | | |
| Eucalyptus Mint | — | 20 | — | 20 | 20 | 5 |
| Vanilla | — | — | 20 | — | — | — |
| Carrageenan | — | — | — | 0.5 | — | — |
| CaHPO₄ | 0.5 | 0.5 | 0.5 | 0.5 | 0.5 | 0.5 |
| Glucono delta- | 0.5 | 0.5 | 0.5 | 0.5 | 0.5 | 0.5 |
| lactone | | | | | | |
| Water | 77 | 77 | 77 | 76.5 | 77 | 92 |

First, a 3 wt. % solution of methylcellulose in water was prepared by weighing methylcellulose into a beaker and carefully pouring in distilled water at a temperature of 95° C. The mixture was stirred for 5 min at 1100 rpm, then cooled first in tap water and then in ice water. Afterwards, the solution was stirred for 10 to 15 min at 1100 rpm.

The alginate or alginate/carrageenan solution (Batch 4) was prepared by adding salt under stirring into water and stirring was continued at 1500 rpm for 2 min at 20° C. The solution was heated to 90° C. and stirred at 2000 rpm for 5 min. Then, the solution was cooled in ice water under stirring at 2000 rpm for 10 min.

Corresponding amounts of the methylcellulose solution and alginate or alginate/carrageenan solution to achieve the concentrations as specified in Table 1 were combined and mixed under stirring at 2000 rpm for 10 min. The fragrance oil was added to the methylcellulose solution and dispersed. A 20 mmol/l solution of GDL in water was added and stirring was continued at 2000 rpm for 5 min.

All samples were well emulsified, which produced smooth compositions with no oily surface, and all gelled at room temperature within 3 hours.

The samples were put into water, and no fragrance oil was observed in the water, demonstrating that the fragrance is stably encapsulated by the compositions. Furthermore, all gels demonstrated considerable strength, elasticity, and moldability.

Other than Batch 4, no syneresis was observed for each sample at 55° C. Batch 4 had low heat resistance, presumably due to the carrageenan, but did contribute to greater hardness for the gel. Syneresis was determined by measuring the amount of water lost or the decrease in the weight of the sample during heating. The heat-resistance for Batches 1-3 was tested by storage in 75° C. for 1 hour, with no syneresis observed. At 75° C. after 8 hours, Batches 1-3 showed significant syneresis (33%, 44%, and 34%), however, upon testing, Batch 6 only showed 8% syneresis.

Batches 1-3 were sliced to form 2 gram cubes, then incubated at 50° C. for 8 hours, and then at room temperature for two weeks. Upon testing by five trained panelists, it was determined that Batches 1-3 were still releasing fragrance (as recognized by human beings in a one meter area), demonstrating long lasting release behavior.

### Example 2

### Comparative

Compositions outside of the present invention are described in Table 2, having the components listed in wt %.

TABLE 2

| | Com- | | | |
|---|---|---|---|---|
| | parative | Comparative | Comparative | Comparative |
| | Batch A | Batch B | Batch C | Batch D |
| METHOCEL A4M | 0.8 | — | 0.5 | 0.5 |
| Methylcellulose | | | | |
| Alginate | — | 1 | — | — |
| Konjac gum | 0.2 | — | — | — |
| Curdlan | — | — | 0.2 | — |
| Guar gum | — | — | — | 0.2 |
| CaCl₂ | — | — | 0.4 | — |
| CaHPO₄ | — | 0.18 | — | — |
| Glucono delta- | — | 0.4 | — | — |
| lactone | | | | |
| Water | 99 | 98.4 | 98.9 | 99.3 |

Comparative Batches A, C, and D used methylcellulose in combination with konjac gum, curdlan, or guar gum, but do not include alginate which is an essential component. The batches displayed phase separation, and the incompatibility could not be solved by adjusting the salt content or blending ratio.

Comparative Batch B forms a gel that is too hard, and would be subject to syneresis, thereby no having controlled release of fragrance over time.

### Example 3

Compositions of the present invention are described in Table 3, having the components listed in wt %.

TABLE 3

| | Batch | | | | | Batch |
|---|---|---|---|---|---|---|
| | 7 | Batch 8 | Batch 9 | Batch 10 | Batch 11 | 12 |
| Carboxy | 1.3 | 1.3 | 1.2 | 1.4 | 1.25 | 1.0 |
| methylcellulose | | | | | | |
| Alginate | 0.9 | 0.7 | 1.0 | 1.0 | 1.25 | 1.25 |
| King Car | 5 | 5 | 5 | 5 | 5 | 5 |
| Fragrance | | | | | | |
| CaHPO₄ | 0.5 | 0.5 | 0.5 | 0.5 | 0.5 | 0.5 |
| Glucono delta- | 0.5 | 0.5 | 0.5 | 0.5 | 0.5 | 0.5 |
| lactone | | | | | | |
| Water | 91.8 | 92 | 91.8 | 91.6 | 91.5 | 91.75 |

Batches 7-12 are made substantially according to the protocol of Example 1. They are tested for water loss (at 75° C. for 8hrs) and hardness (by texture analyzer) with the results listed in Table 4:

TABLE 4

| | | | | | | Batch |
|---|---|---|---|---|---|---|
| | Batch 7 | Batch 8 | Batch 9 | Batch 10 | Batch 11 | 12 |
| Water loss | 6.7 | 8.0 | 7.5 | 6.0 | 6.3 | 7.7 |
| (wt. %) | | | | | | |
| Hardness (g) | 651.3 | 326.3 | 688.2 | 603.8 | 1146 | 975.7 |

Depending on the ratio of alginate to CMC, and relative amounts, water loss and hardness varies. By using this information, optimized formulations can be achieved.

1. An aqueous gel composition for forming a fragrance-containing gel, comprising:
from 0.02 to 5 wt. % of a gel network blend, comprising:
a) methylcellulose, hydroxypropyl methylcellulose, carboxymethylcellulose, or mixtures thereof, and
b) alginic acid, alginates, or mixtures thereof, in a weight ratio ratio of from 1:8 to 8:1;
from 1 to 40 wt. % of a fragrance oil; and
from 0.01 to 10 wt. % of a salt, thereby cross-linking the alginic acid, alginates, or mixtures thereof;
provided that fragrance-containing gel is heat resistant up to 50° C.

2. The composition of claim 1, wherein the gel network blend is present in a range from 1.5 to 3 wt. %.

3. The composition of claim 1, the weight ratio of methylcellulose, hydroxypropyl methylcellulose, carboxymethylcellulose, or mixtures thereof to alginic acid, alginates, or mixtures thereof is 1:1.

4. The composition of claim 1, further comprising glucono delta-lactone, and wherein the salt is selected from calcium phosphate, calcium hydrogen phosphate, and mixtures thereof

5. The composition of claim 1, wherein the aqueous gel composition is substantially free of curdlan, guar gum, fenugreek gum, locust bean gum, konjac gum, agarose, or mixtures thereof

6. The composition of claim 1, further comprising curdlan, guar gum, fenugreek gum, locust bean gum, konjac gum, agarose, or mixtures thereof

7. The composition of claim 1, comprising greater than 70 wt. % water.

8. The composition of claim 1, comprising 20 wt. % fragrance oil.

9. An air freshener made from the composition of claim 1.

10. An air freshener made from the composition of claim 8.
